(19) United States
(12) Patent Application Publication
(10) Pub. No.: US 2026/0269016 A1
(43) Pub. Date: Sep. 10, 2026

(54) AUTOMATED AND QUANTITATIVE INTERPRETATION OF ANTIBODY PROFILES

(71) Applicants: ASSISTANCE PUBLIQUE HOPITAUX DE PARIS, Paris (FR); UNIVERSITE PARIS CITE, Paris (FR)

(72) Inventors: Romain LHOTTE, Gif-sur-Yvette (FR); Jérémy SIEMOWSKI, Thorigny-sur-Marne (FR); Jean-Luc TAUPIN, Paris (FR)

(21) Appl. No.: 19/167,294

(22) PCT Filed: Mar. 19, 2024
(86) PCT No.: PCT/EP2024/057217

§ 371 Date: Sep. 19, 2025

(30) Foreign Application Priority Data
Mar. 20, 2023 (EP) 23305374.3

(51) Int. Cl. G16B 40/00

(52) U.S. Cl. CPC G16B 40/00

(57) ABSTRACT

A method for identifying, in a set of antibody targets, antibody targets corresponding to antibodies in a serum is disclosed. A set of measurement values are obtained by solid phase assays. A measurement value obtained for an antigen is representative of an amount of one or more antibodies bound to the solid phase and directed against one or more antibody targets present on the antigen. For each antigen, a subset of antibody targets present on the antigen is obtained. A search of a candidate vector associated with the lowest error is performed. A candidate vector includes a component for each antibody target in the set of possible antibody targets and a component represents a quantitative contribution to a measurement value of the corresponding antibody target. The error associated with a candidate vector is based on a sum of squared antigen errors across the set of antigens. An antigen error is obtained by comparing the sum of quantitative contributions in the candidate vector with the measurement value obtained for the concerned antigen. The antibody targets corresponding to the non-zero components in the candidate vector associated with the lowest error are identified.

### TECHNICAL FIELD

Various example embodiments relate generally to automated and quantified interpretation of antibody profiles. More precisely a method for identifying, in a set of antibody targets, one or more antibody targets corresponding to one or more antibodies in a serum and a corresponding computing device are disclosed.

### BACKGROUND

Graft rejection is one of the most important complications (in severity and frequency) of solid organ transplantation and to a lesser extent of hematopoietic stem cell transplantation. It is most often caused by antibodies (DSA, donor-specific antibody) produced by the recipient and directed against the donor's HLA (human leukocyte antigen) system, capable of destroying the cells of the graft, in an acute (rapid) or chronic (slow and progressive) mode. These anti-HLA antibodies are either pre-existing in the plasma (following pregnancies, transfusions, and/or previous transplants) or produced de novo after the transplant (for organ transplantation, either induced by the graft or induced by the same causes as the pre-existing antibodies).

The HLA system comprises genes encoding class I proteins (among which the A, B and C genes) composed of one HLA gene product, and class II proteins (among which the DR, DQ and DP genes) that are heterodimers of two HLA gene products (DRA and DRB etc. . . . ). The HLA system is extremely polymorphic, totalling currently >30,000 unique genetic variants called alleles, encoding >20,000 proteins differing between each other by at least one gene-encoded amino acid polymorphism. According to the official HLA nomenclature, the genetic variants encoding unique protein sequences are designed as “second field alleles” and immunologically correspond to the HLA antigens. These polymorphisms cause differences between donors and recipients, that are called eplets when they are able to trigger anti-HLA antibody and DSA formation. In return, each of the antibodies and DSA produced by the immune response binds to a small part called the epitope of the HLA antigen. The epitope grossly surrounds and also includes the eplet. An HLA antigen is therefore a unique mosaic of eplets and epitopes embedded in a more or less conserved overall structure and conformation, and shared between variable numbers of alleles. For class I the antigen is the product of the class I allele but for class II it is the combination of the products of the two class II alleles, because only the combination can be produced by the cell in a functional form, that is capable of triggering the immune response, each gene product being unstable when produced alone. Antigens that are close enough define one given serological group, and all HLA antigens are supposedly linked to one of the around 150 serological groups that have been defined over time. A serum may contain more than one antibody or DSA targeting a single to many HLA serological group(s) via the recognition of between one to many eplets.

Solid phase assays are used for antibody identification for a set of antigens adsorbed on the solid phase and incubated in a serum of a recipient patient and measurement values are obtained. A measurement value that is obtained for an antigen is representative of an amount of one or more antibodies directed against one or more antibody targets present on the corresponding solid phase.

As an example, the antibody profiles may be determined using an antigen fluorescence kit which analyses the reactivity of a patient's serum against nearly 200 of the most frequent antigens of the HLA system. A set of solid phase particles (e.g., beads) are incubated in the serum, where each bead is covered with antigen molecules produced from a single allele for HLA of class I or of a single pair of alleles for class II. A fluorescence measure (e.g., a MFI, Mean Fluorescence Intensity) is obtained for each bead after separation from the serum, a measurement value being representative of an amount of antibodies directed against one or more antibody targets present in the antigen molecule on the bead. A single MFI value does not allow deciphering the number of and which eplet(s) are targeted by the serum, but the global interpretation of the whole beads panel is supposed to make this possible.

The interpretation of the measurement values is currently carried out mainly qualitatively (i.e. the bead is considered positive or negative) to determine whether the antibodies and DSA are present or absent, and the results are mostly interpreted manually by biologists. This may be time consuming when the number of sera to be analysed is high (for example, between 1,000 and 50,000 per year per HLA class in France depending on the laboratory size) and also when the sera profiles are complex (i.e. when many beads are positive).

Different types of antibody targets may be considered for the interpretation. At a first interpretation level, only antibodies directed against whole serological groups or specific to antigens may be considered by the biologist. In this situation, all the beads bearing close enough alleles are dependent from each other, and each bead is independent from all the others. At a second level, eplets may be considered but in order to reach this level of interpretation, the required time may be important and this level is very rarely used because usually very complex. Therefore, three categories of antibody targets can be defined, from the least to the most precise in terms of identification of the antibody binding site on the HLA protein: the serological group, the antigen and the eplet. Indeed, any given eplet being usually distributed over several to many beads, the MFI value measurement for any bead may represent the sum of individual MFI values from an a priori undetermined number of eplets, complicating eplet identification.

There exists an automated eplet interpretation tool called HLA Matchmaker®) that is considered the gold standard to reach the eplet level of interpretation, but it only allows to approach it qualitatively. Indeed, this tool provides the list of eplets that are present on all the beads classified by the user as positive, and only on those beads, without considering the individual MFI values of the truly explicative eplet(s). Therefore, it provides in most cases an excess of possible solutions to explain an antibody profile. In addition, it only addresses the problem qualitatively, i.e., by simply naming eplets possibly involved in the serum reactivity and excluding those that are expressed on at least one bead classified as negative.

In order to reduce the immunological risk for the recipient (i.e. the possibility that the recipient's antibodies recognize the donor's antigens), the interpretation made manually by the biologist of the recipient's serum profile is naturally and involuntarily biased towards a tendency to declare an excess of anti-HLA antibodies (pre-transplant, in order to define the spectrum of donors not recognized by the recipient's sera) and therefore DSA (at time of and post-transplant), when a donor has been selected. This excess may lead, pre-transplant, to a reduction in access to transplantation for patients, and, post-transplant, to an increase in immunosuppressive treatment to control antibodies erroneously considered as DSA. Therefore, improving anti-HLA antibody identification would improve patient management.

Moreover, as the interpretation is mainly carried out manually, with for example 200 measurement values per serum (e.g., with one fluorescence value per bead), the repeatability and reproducibility of the interpretations are not guaranteed and human errors or subjective interpretations are inevitable.

### SUMMARY

The scope of protection is set out by the independent claims. The embodiments, examples and features, if any, described in this specification that do not fall under the scope of the protection are to be interpreted as examples useful for understanding the various embodiments or examples that fall under the scope of protection.

According to a first aspect, a method for identifying, in a set of antibody targets, one or more antibody targets corresponding to one or more antibodies in a serum is disclosed. The method comprises
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen;
(d) identifying one or more antibody targets corresponding to one or more non-zero components in a candidate vector associated with the lowest error and extracting the non-zero components corresponding to the quantitative contributions of the identified one or more antibody targets.

In one or more embodiments, searching in the multidimensional search space the candidate vector associated with the lowest error comprises:
(c1) initializing candidate vectors forming an initial mesh;
(c2) repeating iteratively the following steps until at least one convergence criterion is met:
   (c21) for each candidate vector, computing the associated error;
   (c22) selecting candidate vectors with the lowest errors;
   (c23) generating new candidate vectors based on the selected candidate vectors, the new candidate vectors forming a refined mesh in the multidimensional search space;
(c3) selecting the candidate vector with the lowest error.

In one or more embodiments, the method further comprises: eliminating from the set of measurement values each measurement value being below a detection threshold and eliminating from the set of antibody targets the one or more antibody targets present on at least one of the antigens for which the measurement value is below the detection threshold before performing step (c1).

In one or more embodiments, the method further comprises eliminating each candidate vector including at least one component whose value is below a positivity threshold but non-zero, before performing step (c21) or (c22).

In one or more embodiments, the method further comprises: identifying, in the set of antigens, independent groups of antigens such that there is no antibody target present on an antigen in a group that is also present on another antigen in another group; and performing the steps (c) and (d) independently for each of the independent groups of antigens.

In one or more embodiments, the method further comprises: adding a penalty factor to the sum of squared antigen errors, wherein the penalty factor increases as a function of the number and/or the value of the one or more non-zero quantitative contributions of the antibody targets in a candidate vector.

In one or more embodiments the penalty factor is computed using a subadditive function of the candidate vector.

In one or more embodiments the penalty factor is relaxed by: executing steps (c) and (d) with the penalty factor to identify the non-zero components of the candidate vector with the lowest error; and repeating steps (c) and (d) without the penalty factor with the candidate vectors being reduced to the identified non-zero components to determine a second candidate vector with the lowest error that gives the values of the identified non-zero components.

In one or more embodiments, the candidate vectors forming the initial mesh in the multidimensional space have a uniform spatial distribution.

In one or more embodiments, the candidate vectors forming the refined mesh in the multidimensional space have a uniform spatial distribution.

In one or more embodiments, a first convergence criterion is met when the elapsed time for executing step (c2) is greater than a threshold.

In one or more embodiments, a second convergence criterion is met when the cell dimensions of the smallest cells of the refined mesh reach a predefined limit.

The method may be a computer-implemented method. According to a second aspect, a computing device comprises means for performing a method according to the first aspect is disclosed. In one or more embodiments, the means comprise: at least one processor; at least one memory storing instructions that, when executed by the at least one processor, cause the computing device to perform the method. The input data may be obtained from a memory of a computing device. Likewise, each subset of one or more antibody targets present on an antigen in the set of antigens may be obtained from a memory of a computing device.

The computing device may comprise means for performing one or more or all steps of the method according to the first aspect. The means may include circuitry configured to perform one or more or all steps of a method according to the first aspect. The means may include at least one processor and at least one memory storing instructions that, when executed by the at least one processor, cause the computing device to perform one or more or all steps of a method according to the first aspect.

According to another aspect, a computing device comprises at least one processor and at least one memory storing instructions that, when executed by the at least one processor, cause the computing device to perform:
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen;
(d) identifying one or more antibody targets corresponding to one or more non-zero components in a candidate vector associated with the lowest error and extracting the non-zero components corresponding to the quantitative contributions of the identified one or more antibody targets.

The instructions, when executed by the at least one processor, may cause the computing device to perform one or more or all steps of a method according to the first aspect.

According to another aspect, a computer program comprises instructions that, when executed by a computing device, cause the computing device to perform the method according to the first aspect.

According to another aspect, a non-transitory computer-readable medium comprising program instructions stored thereon for causing a computing device to perform:
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen;
(d) identifying one or more antibody targets corresponding to one or more non-zero components in the candidate vector associated with the lowest error and extracting the non-zero components corresponding to the quantitative contributions of the identified one or more antibody targets.

The program instructions may cause the computing device to perform one or more or all steps of a method according to the first aspect.

### BRIEF DESCRIPTION OF THE DRAWINGS

Example embodiments will become more fully understood from the detailed description given herein below and the accompanying drawings, which are given by way of illustration only and thus are not limiting of this disclosure.

FIG. 1 is a schematic diagram illustrating relationships between data used by the algorithm according to an example.

FIG. 2 shows a flowchart of a method for identifying, in a set of antibody targets, one or more antibody targets corresponding to one or more antibodies in a serum according to example embodiments.

FIG. 3 shows a flowchart of a method for searching in the multidimensional search space a candidate vector associated with the lowest error according to example embodiments.

Each of FIGS. 4A-4F shows a table including experimental results obtained for a simplified case according to an example.

FIG. 5 shows a table with experimental results obtained for an example another case.

FIG. 6 is a block diagram illustrating an exemplary hardware structure of a computing device according to an example.

It should be noted that these drawings are intended to illustrate various aspects of devices, methods and structures used in example embodiments described herein. The use of similar or identical reference numbers in the various drawings is intended to indicate the presence of a similar or identical element or feature.

### DETAILED DESCRIPTION

Detailed example embodiments are disclosed herein. However, specific structural and/or functional details disclosed herein are merely representative for purposes of describing example embodiments and providing a clear understanding of the underlying principles. However these example embodiments may be practiced without these specific details. These example embodiments may be embodied in many alternate forms, with various modifications, and should not be construed as limited to only the embodiments set forth herein. In addition, the figures and descriptions may have been simplified to illustrate elements and/or aspects that are relevant for a clear understanding of the present invention, while eliminating, for purposes of clarity, many other elements that may be well known in the art or not relevant for the understanding of the invention.

Solid phase assays are used for antibody identification for a set of antigens adsorbed on the solid phase and incubated in a serum of a recipient patient and measurement values are obtained. Any technology may be used for obtaining these measurement values.

According to an example technology, antibody profiles may be determined using a single antigen fluorescence kit that separately analyses the reactivity of the recipient's serum against about 200 of the most common HLA antigens, each being coated on a distinct bead and each carrying a given number and list of eplets according to its gene sequence.

Amino acid sequence variations between the >20,000 known HLA proteins are the cause of recipients' immunization, when these HLA proteins are provided by the donors, as they create “non-self” recognized as such by the recipients' immune system. In return, the immune system will generate one or more DSA specifically directed against one or more of these different regions within the entire donor HLA protein. These small regions of one or a few surface accessible amino acids are called “eplets” and are part of the epitopes, these epitopes being the actual targets of antibodies.

The recipient serum is incubated with the bead mixture and then with a fluorescent conjugate directed against the human immunoglobulin isotype of interest (usually IgG, but also IgM or other). The fluorescence (e.g., MFI) measured after incubation of the recipient serum and then the conjugate is a reflection of the reactivity of the patient's serum against this antigen.

If one or more anti-HLA antibodies are present in the serum, one or more beads become fluorescent, i.e., are classified as “positive”. The fluorescence of the conjugate measured for each bead makes it possible to conclude on the reactivity of the antibodies in the recipient's serum against such or such HLA antigen, both qualitatively (positive if the fluorescence is higher than a positivity threshold, for example set at 500 fluorescence arbitrary units) and quantitatively on the concentration/affinity (a combination of parameters defining the “strength”) of the antibody in the serum according to the value of the positive fluorescence measured (possible range between 500 and 25000 units, per bead). The positivity threshold may be understood as a reactivity threshold that allows to determine whether or not the antibody is reactive or not to an antibody target.

The complexity of the HLA system (around two dozen genes per patient with for each of them a plethora of allelic variants, leading to an enormous diversity of donor/recipient combinations) makes the interpretation of these antibody profiles often complex. This complexity is at the origin of errors in the identification of these antibodies and DSA, which can lead to a misrecognition of a DSA before or after transplantation.

At any given time, a patient may have produced one or more antibodies to a variable number of different eplets, but remains unable to produce antibodies to their own antigens (and therefore their own eplets). An antibody can recognize a very large number of different antigens, if the target eplet is very highly represented in the kit. But the situation is often more complex, especially in the following two cases:
1) when the HLA protein consists of 2 polymorphic protein chains, as are the DQ and DP class II proteins. An antibody can recognize either of the two chains, or even particular eplets generated at the contact zone of by particular combinations of these two chains.
2) when several antibodies coexist against different eplets of the same HLA protein: the MFI measured against this antigen, is then, in the case of two antibodies, composed of x % of an antibody directed against an A eplet and (100-x) % of an antibody directed against a B eplet, and even more than two antibodies can be present. It therefore becomes very difficult to interpret these profiles manually, because of their complexity and also because of the time needed to explore a variable number of possibilities, without any guarantee of success, i.e., without any real objective criterion for selecting the most probable combination. In addition, when the interpretation is performed by several biologists independently, the results are different in many cases (either on the identity of the targets recognised by the antibodies, or on their individual MFI, or on both criteria).

The knowledge of the “strength” of each antibody identified as a DSA, makes it possible to measure the immunological risk to the recipient: the higher the strength, the greater the risk. This makes it possible to accept/reject a graft and after the graft to modulate immunosuppression to avoid rejection.

A method is disclosed therein that is configured to autonomously determine the contributions to the measurement values of a set of antibody targets, with a high precision and finesse. The method is based on an algorithm that allows a quantitative analysis at various analysis levels (including the serological group, the antigen and/or the eplet levels). The algorithm adapts to any number and any type of antibody targets. The algorithm also adapts to any addition, removal or change of serological groups/antigens/eplets that may be done by a user.

Each antigen is associated with a list of candidate antibody targets, which is taken into account by the algorithm to determine both the qualitative aspect (presence/absence of antibody/ies) and the quantitative aspect (contribution of the present antibody/ies directed against each of the candidate antibody targets to the measurement value obtained for the antigen). For example, a given antigen may be characterized by the antigen itself, the serological group it belongs to, and a list of eplets the antigen contains: this defines the list of candidate antibody targets for the given antigen that the algorithm is allowed to identify. The association between an antigen and its list of antibody targets may be updated as necessary, for example to follow the evolution of knowledge concerning this antigen (e.g., discovery of new eplets or removal of falsely previously identified eplets, etc).

The algorithm is configured to determine the respective quantitative contributions to the measurement value (MFI) of each of one or more antibodies directed to one or more candidate antibody targets present on each antigen in the serum.

The algorithm allows the analysis of the measurement values for a serum in a very short time, e.g., on average in less than a few seconds on a recent processor. The algorithm allows to process the measurement values at high speed, in the order of 100 to 10,000 sera per hour on a microcomputer with 8 GB of RAM using the 4 processor cores running in parallel.

The algorithm identifies a set of one or more antibodies in the recipient serum (qualitative assessment) and calculates their respective “strength”, i.e., the proportion of measurement value to which each antibody contributes to the overall measurement value obtained for an antigen. This quantitative assessment takes into account the expected consistency across all measurement values obtained for all the antigens (e.g., all MFI values of all the positive beads).

The algorithm is based on a system of linear equations, whose unknowns are the quantitative contributions of the antibody targets to the measurement values for the antigens.

However, the searched space is very large, of the order of ~25000^{e} with 25,000 being the range of possible MFI values that can be measured with a step of 1 unit. “e” is the number of antibody targets, usually several hundreds, depending on the category/list of antibody targets chosen by the user when the interpretation (antigen and serological groups, only some eplets, all eplets, etc. . . . ) of the MFI profile is run according to the objective of the interpretation to be performed and depending on the degree of finesse or resolution to be reached.

In order to measure the proportion of MFI attributable to each antibody, the algorithm is configured to explore all possible combinations of quantitative contributions of the antibody targets attached to the positive antigens, in order to obtain the most accurate (i.e., the smallest error) solution (i.e., a set of antibody targets with their respective MFIs, which are considered the most probable and constitute the result of the interpretation of the profile).

The exploration of all possible combinations of quantitative contributions may be done using candidate vectors defined in a multidimensional space whose dimension may correspond to the number of considered antibody targets. The candidate vectors may define an initial mesh that is refined iteratively until one or more convergence criteria are met. The candidate vectors forming the initial mesh and/or the refined mesh may have a uniform spatial distribution.

Mathematically, this optimal solution minimizes the sum of the squares of the deviations between the values (e.g., MFI) predicted by the algorithm and the measurement values (e.g., MFI) obtained for the antigens. The deviations may be weighted with a weight function to compensate for the shortcomings of measuring absolute squared deviations. The antibody targets identified by the algorithm are those that best explain the entire serum, i.e., all the measurement values, taking into account all antibody targets belonging to the category/list of antibody targets chosen by the user at the time of interpretation, on all tested antigens.

As the system of equations has more unknowns than equations, the simplest and most biologically probable solutions are preferred. This can be done by adding a penalty to the least probable solutions (e.g., using a penalty factor, as described below) in a Lasso/Ridge manner except the ½-norm is used.

The algorithm may exclude one or more antibody targets that are unnecessary for the interpretation of the measurement value: if a set of one or more antibody targets belonging to the recipient is provided as input to the algorithm, the algorithm may be configured to exclude the set of one or more antibody targets of the recipient from the considered set of antibody targets.

Groups of antigens that are independent of the others may be processed independently by the algorithm. These independent groups are such that there is no antibody target present on an antigen in a group and also present in another antigen in another group.

A graph or tree structure of antibody targets from most to least covering (i.e., covering from the most to the least number of antigens) may be generated and used to limit the searched set of antibody targets: with the algorithm processing the most covering ones first, and integrating the least covering ones if the most covering ones are not sufficient to explain the serum. By combining this with recursive methods, i.e., capable of finding local optimums (for example, n=2, 5, 8, 10, 12, 15, etc) and then reiterating from these local optimums until the global optimum is found, the search time is drastically reduced.

The penalty factor is a means for performing regularization. However, although the number of selected antibody targets (non-zero values in the selected vector) is reduced, their quantitative contributions are biased towards zero. A two-stage procedure, termed relaxation, overcomes the bias side-effect. The first procedure controls the variable selection, as in ordinary Lasso/Ridge estimation, and the second procedure repeats the first one without the penalty factor but only allowing the antibody targets returned by the first procedure, the quantitative contributions are therefore selected sparsely and are not biased.

It is to be noted that the algorithm is adapted to process any type of measurement values (not only MFI) that may be obtained using any type of solid phase assays.

Other possible solid-phase assays that include any multiplex test (analyzing a panel of targets either individualized or mixed) having a quantitative reading (fluorescence, luminescence, optical density, etc.) will be able to benefit from the algorithm. For example, antigen/eplet identification in IgE-mediated immediate hypersensitivity to all kinds of allergens, antigen/eplet identification in antibody-mediated immunization to pathogens (e.g., viruses) prone to genetic variation, or antigen/eplet identification on non-protein compounds such as lipids, sugars or synthetic products. More generally, the algorithm may be applied to all situations dealing with a variety of antibody targets (being proteins, sugars, lipids or any other biological or chemical compound) with potential cross-reactivity (i.e. sequence/structure variations close enough to maintain antigen/antibody interaction with more than one of the considered targets), with a cognitive or medical interest.

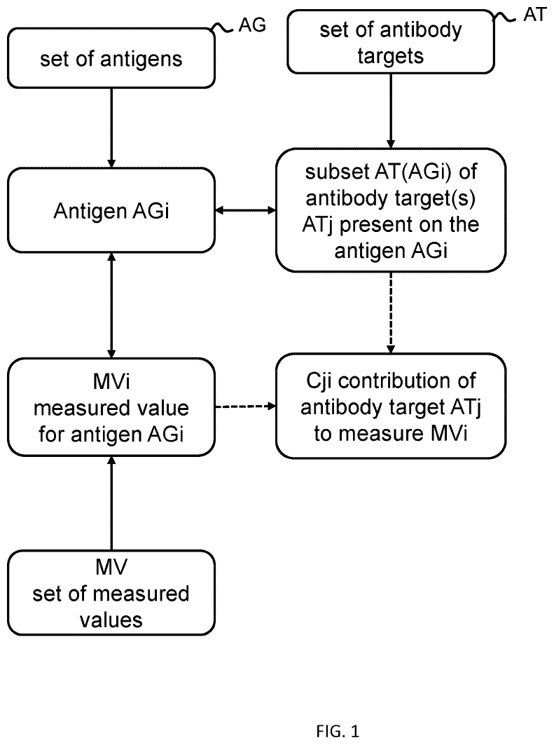

FIG. 1 is a schematic diagram illustrating relationships between data used by the algorithm according to an example.

The set of measurement values is noted MV, and a measurement value for an antigen is noted MVi (i being the antigen index). When beads are covered with class I HLA antigens, each bead carries an antigen with the name of the corresponding second field allele cited according to the international nomenclature. For HLA class II, two second field alleles encode for the antigen, one for the alpha chain (e.g., DQA1*02:01) and one for the beta chain (e.g., DQB1*03:01).

A measurement value (MVi) is obtained for each antigen, in the form of a MFI. An MFI value represents the “strength” of the antibody(s) present in the serum against the antigen(s).

The set of tested antigens is noted AG. In the set of measurement values MV, a measurement value MVi is obtained for an antigen AGi in the set of tested antigens AG. The set of antibody targets is noted AT and includes all antibody targets present on the tested antigens. For each antigen AGᵢ in the set of tested antigens AG, a subset AT(AGᵢ) of one or more antibody targets ATⱼ (j being the antibody target index) present on the antigen AGi is provided as input data to the algorithm. Let Cᵢⱼ be the contribution of the antibody target ATⱼ to the measurement value MVi such that MVi=ΣⱼCᵢⱼ assuming here that Cᵢⱼ is always zero if the antibody target ATⱼ is not present on the antigen AGᵢ. The contributions are the unknowns to be found by the algorithm.

There are several ways to define the subset AT(AGᵢ) of one or more antibody targets of an antigen AGᵢ.

The antibody targets that the algorithm is allowed to identify, and that are associated with each antigen, provide the possible explanations for the measurement value obtained for this antigen, knowing that one or more antibodies recognizing as many different antibody targets on the same antigen can be present at the same time in a serum and therefore add up to the MFI. These antibody targets may include serological groups, antigens, eplets or a mixture of at least two of these three categories. Depending on the level of precision required, a user may use any list of antibody targets adapted to his needs. For example, for a “clinical” level result, i.e. understandable by the clinician who follows a patient, the serological group+antigen level corresponds to the current clinical definition of compatibility as it is used by the governmental agencies managing organ allocation nationwide in most countries worldwide. For a “scientific” interpretation, the eplet level is closer to the reality of the immune response and is currently gaining in interest clinically. The epitope level is the ideal level to reach, but the current knowledge about the antigen/antibody interaction is too preliminary to be applied reliably to patients. It is recommended to run the algorithm separately for the “clinical” and the “scientific” levels, because merging several lists of antibody targets renders the task more complex for the algorithm and brings confusion in the output as two conceptions of HLA compatibility are being mixed together. The list of antibody targets may also grow over time as discoveries are made by biologists, especially by considering epitopes at the pace they will be characterized in the future. For eplets, the list of antibody targets fluctuates, and some may disappear if they cannot be confirmed experimentally.

For example: DEAV or VGPM/GGPM are eplets that occupy the same location (amino acids 84, 85, 86 and 87) on the DP beta chain and are therefore never present together on the same DP molecule.

NB: the notations used herein are based on the international nomenclature, except for DP for which serological groups and antigens have not been strictly defined. In this document, DP*xx and DPAxx represent the DP beta and alpha chains, respectively, and xx designates a serological group.

The subsets of antibody targets to be used for the antigens may be stored in a database mapping an antigen with a subset of one or more antibody targets and be used as necessary for several sera.

A user may modify this mapping as needed. For example, there may be one or more eplets which the user wishes to take into account on the basis of personal experience, even if they are not officially described. This is the case for the DQ eplets associating specific combinations of alpha and beta chains.

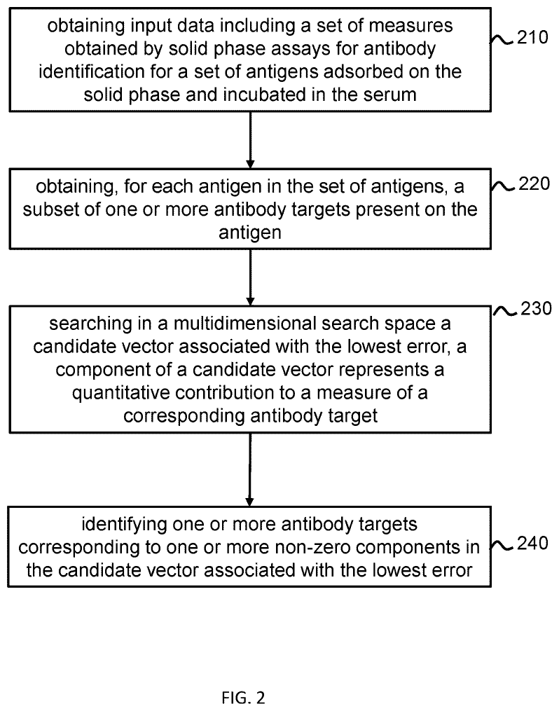

FIG. 2 shows a flowchart of a method for identifying, in a set of antibody targets AT, one or more antibody targets corresponding to one or more antibodies in a serum according to example embodiments. The steps of the method may be implemented by a computing device as described herein.

While the steps are described in a sequential manner, the person skilled in the art will appreciate that some steps may be omitted, combined, performed in different order and/or in parallel.

In step 210, input data are obtained: the input data include a set of measurement values MV obtained by solid phase assays for antibody identification for a set of antigens adsorbed on the solid phase and incubated in the serum. A measurement value MVi obtained for each antigen AGi is representative of the strength of one or more antibodies directed against one or more antibody targets present on the antigen. A measurement value may be for example an MFI value.

The set of measurement values MV may be a set of preprocessed measurement values generated from raw measurement values. For example, one or more preprocessing operations may be performed depending on the type of technology used to generate the raw measurement values. In the case of MFI measurement, the preprocessing operations may include: suppressing background signal; correcting non-linear effects; etc.

Further, in order to reduce the processing time, each measurement value being below a first threshold, referred to herein as the detection threshold, may be eliminated from the set of measurement values MV: this detection threshold allows to eliminate all non-significant measurement values, e.g., corresponding only to noise. The measurement values that are below the detection threshold are considered as being non-significant measurement values. The detection threshold may be an empirical threshold agreed upon by biologists and that follows recognized recommendations or a dynamic threshold varying from the recommended one. In a dynamic configuration, the threshold autonomously adjusts for each serum sample, based on an estimation of the extent to which said serum is influenced by extraneous interference. As a consequence, the set of antibody targets used as input may then be reduced by keeping only those present only on antigen(s) for which the measurement values are above the detection threshold before performing step 230. This means that if an antibody target is present on at least one antigen that has a measurement value below the detection threshold, then the antibody target is eliminated.

In step 220, for each antigen AGᵢ in the set of antigens, a subset AT(AGᵢ) of one or more antibody targets present on the antigen AGᵢ is obtained.

In order to further reduce the processing time, the set of antigens used for performing next steps 230-240 may be divided into independent groups of antigens and steps 230-240 be performed on each independent group of antigens separately. As explained above, two groups of antigens are independent if there is no antibody target present on an antigen in a first group that is also present on another antigen in a second group. The groups may be identified by using any classification method (for example based on a graph) that allows to determine if a given antibody target has to be classified in an existing group or if a new group has to be generated with this antibody target.

The independent groups of antigens may therefore be identified in the set of antigens before execution of steps 230 and 240: the set of antigens (and the corresponding set of measurement values) used for steps 230 and 240 corresponds to one of the independent group of antigens. As a consequence, the set of antibody targets AT to be considered for the execution of steps 230 and 240 may therefore be a reduced set of antibody targets AT that includes only those present on at least one antigen of the group of antigens to which steps 230 and 240 are applied. This will have the effect of reducing the dimension of the multidimensional search space and of the candidate vectors.

In step 230, a search for a candidate vector associated with the lowest error is performed in a multidimensional search space.

A candidate vector includes a component for each antibody target ATⱼ in the set of antibody targets AT. A component of a candidate vector represents a quantitative contribution to a measure of the corresponding antibody target ATⱼ. The error associated with a candidate vector may be based on a sum of squared antigen errors across the set of antigens AG. An antigen error may be obtained for an antigen AGi by comparing the sum of quantitative contributions in the candidate vector with the measurement value MVi obtained for the concerned antigen.

In one or more embodiments, a penalty factor may be added to the sum of squared antigen errors. This penalty factor allows to add a penalty to the least probable solutions, such that the simplest and most biologically probable solutions are preferred. The penalty factor may for example increase as a function of the number and/or the value of the one or more non-zero quantitative contributions of the antibody targets in a candidate vector. The penalty factor may be computed using a subadditive function of the candidate vector.

In step 240, one or more antibody targets are identified: the identified antibody targets correspond to one or more non-zero components in a candidate vector associated with the lowest error: these antibody targets correspond to those that effectively contribute to the measurement values and explain these measurement values. The non-zero components of the candidate vector associated with the lowest error are extracted and correspond to the quantitative contributions of the identified one or more antibody targets.

In one or more embodiments, the penalty factor is relaxed by: executing steps 230 and 240 with the penalty factor to identify the non-zero components of the candidate vector with the lowest error; and repeating steps 230 and 240 without the penalty factor with the candidate vectors being reduced to the identified non-zero components to determine a second candidate vector with the lowest error that gives the values of the identified non-zero components.

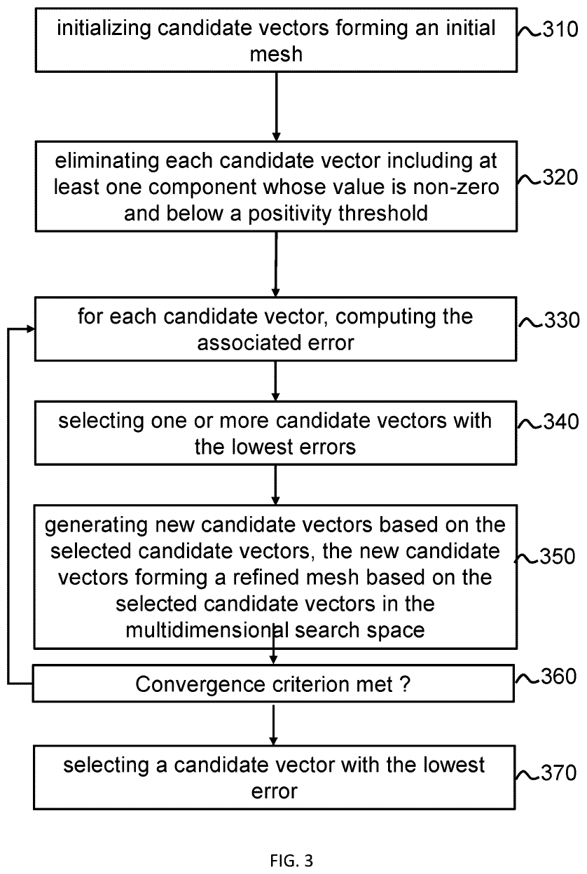

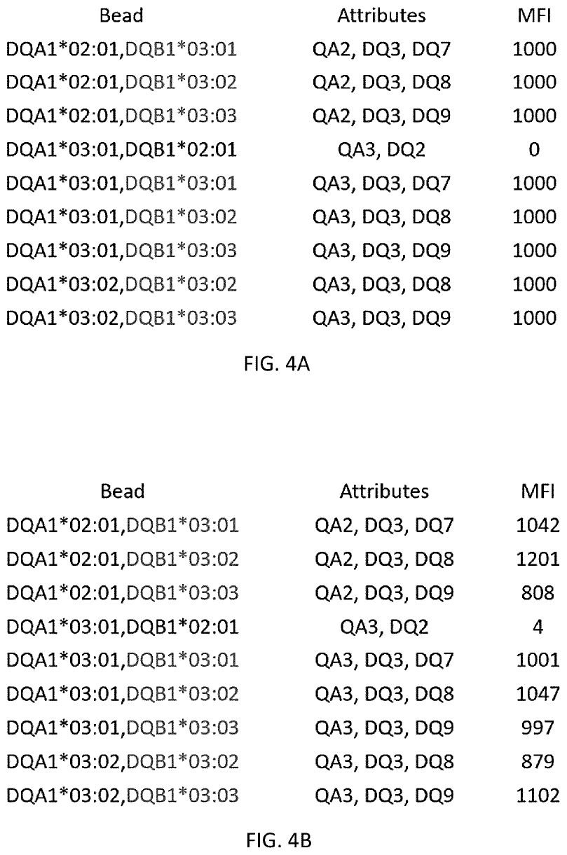

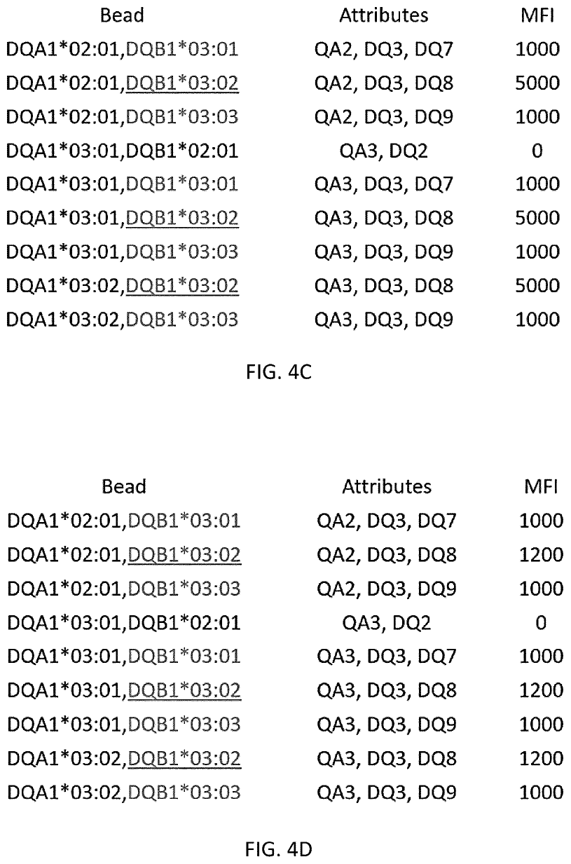

FIG. 3 shows a flowchart of a method for searching in the multidimensional search space a candidate vector associated with the lowest error according to example embodiments.

The steps of the method may be implemented by a computing device as described herein. The steps may be performed during step 230 of the method described by reference to FIG. 2.

While the steps are described in a sequential manner, the person skilled in the art will appreciate that some steps may be omitted, combined or performed in different order and/or in parallel.

In these example embodiments, the search in the multidimensional search space is performed using an initial coarse mesh that is refined iteratively.

In step 310, candidate vectors forming an initial mesh are generated. The candidate vectors forming the initial mesh in the multidimensional space may have a uniform spatial distribution.

In step 320, each candidate vector including at least one component whose value is non-zero and below a positivity threshold may be eliminated and therefore not considered for the search. Similarly to the detection threshold that is applied to a measurement value, the positivity threshold that is applied to a component in a candidate vector may follow recognized recommendations or can be a dynamic threshold inferring its value from the serum's values. This step allows removing too small values that do not correspond to a potential contribution of an antibody target.

In step 330, for each candidate vector, the associated error is computed.

In step 340, one or more candidate vectors with the lowest errors are selected for the next iteration step.

In step 350, the current mesh is refined: new candidate vectors are generated based on the selected candidate vectors, the new candidate vectors forming a refined mesh in the multidimensional search space. Like the initial mesh, the candidate vectors forming the refined mesh in the multidimensional space may have a uniform spatial distribution. The refined mesh may be a mesh refined only based on (e.g., around) the selected candidate vectors where the global minimum is likely to be located, e.g., using a refined mesh with a smaller mesh step or by interpolation of the selected candidate vectors.

Steps 330-350 are executed iteratively until at least one convergence criterion is met. The convergence criteria may be tested in step 360. A first convergence criterion is met when the total elapsed time for all iterations is greater than a threshold. A second convergence criterion is met when the cell dimensions of the largest cells of the refined mesh reach a predefined limit, i.e. the mesh is enough refined. If the at least one convergence criterion is met, then step 370 is executed after step 360. If the at least one convergence criterion is not met, then a new iteration is started by going back to step 330 after step 360.

In step 370, the candidate vector with the lowest error is selected as the output of algorithm and allows to identify the antibody targets that contribute to the measurement values.

The algorithm disclosed by reference to FIGS. 2 and 3 is configured to search a solution to an optimization problem as formulated by the equations below. The optimization problem arises as the biological problem can be translated to an under-determined system of linear equations with noisy observations x.

Let x∈({0}∪[xₘᵢₙ; +∞])^{a} be a candidate vector of dimension a, where a candidate vector includes a component xⱼ with j∈{1, . . . , a} for each antibody target ATⱼ in the considered set of antibody targets AT (the number of antibody targets in AT is therefore equal to the dimension a) and xₘᵢₙ represents the positivity threshold for a component. A component xⱼ represents a quantitative contribution to a measure of the corresponding antibody target ATⱼ.

The goal of the optimization problem is to find a candidate vector x that minimizes the error E(x) defined by equation (Eq1):

$$E ( x ) = {\sum }_{i = 0}^{b} {( {y}_{i} - {β}_{i}^{T} x )}^{2} + g ( x ) \qquad ( Eq 1 )$$

in which:
yᵢ is a measurement value obtained for an antigen identified by index i, with i∈{1, . . . , b}, b being the number of antigens (equal here to the number of measurements values), where the measurement values may be preprocessed measurement values obtained after preprocessing (suppression of background noise, correction of non-linear effects, etc); βᵢ is a vector of binary values of length b, where each component (βᵢ)ⱼ indicates whether the antibody target ATⱼ of index j is present on the antigen i;

$${β}_{i}^{T}$$

is the transpose vector of vector βᵢ;

$${β}_{i}^{T}$$

x is the scalar product of

$${β}_{i}^{T}$$

and x;
g(x) is a subadditive function from [symbol] to [symbol] that defines the penalty factor applied to candidate vector x.

As explained above with respect to FIG. 2, the measurement values below the detection threshold may be eliminated from the considered set of measurement values. This means that in the equation Eq1, the sum may be computed only for the measurement values that are not below the detection threshold. As a consequence, the set of antibody targets to be used as input may also be reduced by eliminating from the set of antibody targets all the antibody targets present on at least one antigen for which the measurement value is below the detection threshold. This detection threshold may vary from one bead to another when MFI values are measured.

The error associated with a candidate vector is based on a sum (computed over the set of antigens) of squared antigen errors

$${e}_{i} = {( {y}_{i} - {β}_{i}^{T} x )}^{2}$$

for i∈{1, . . . , b}.

The above sum given by (Eq1) may be minimized subject to the following constraints C1 and C2:

$$∀ {j}_{1} ∈ \{ 1 , … , a \} , ∃ i ∈ \{ 1 , … , b \}$$

such as

$${( {β}_{i} )}_{{j}_{1}} = 1 \qquad ( C1 )$$

$$∀ {j}_{2} ∈ \{ 1 , … , a \} ∖ {j}_{1} : ( {β}_{i} ) = 1 , \qquad ( C2 )$$

$${x}_{{j}_{1}} \geq - \frac{arc tan ( \frac{( {y}_{i} - 10000 )}{1000} ) + 4.5 )}{4} \times {x}_{{j}_{2}}$$

where:
constraint C1 implies that the antibody target j₁ is present on the antigen i;
constraint C2 implies that for all antibody targets j₂ that are not j₁, the MFI contribution of j₁ is significant enough with regards to other antibody targets.

These constraints may be provided as input to an optimization software. Otherwise, a penalty may be applied to the error associated with the candidate vectors that do not meet the above constraints to increase artificially their respective errors such that the candidate vectors that do not meet the above constraints will never be selected as the candidate vectors with the lowest errors.

The function g(x) returns a penalty factor encouraging sparse attribute distribution which could be compared to what a Lasso/Ridge Regression would do.

Here the penalty function g(x) may be defined by:

$$g ( x ) = {λ}^{3 / 2} {{\sum }_{j = 1}}^{a} \sqrt{{x}_{j}}$$

where

$$λ = ( {min}_{j ∈ \{ 1 , … , a \}} {P}_{j} ) - 10$$

where Pⱼ is the positivity threshold for antibody target j.

The more antibody targets are used to describe the serum, the bigger the penalty. But with a L₀ norm, the penalty would never exceed

$${( {min}_{j ∈ \{ 1 , … , a \}} {P}_{j} )}^{2}$$

otherwise, the effect of the positivity threshold would be overridden by this penalty. And this upper limit is a problem since it would be negligible for large fluorescence values, and therefore too many antibody targets would be involved to describe the serum.

However, since the optimization problem is not only consisting of an ordinary least squares term, the best solution is not the mean function, g(x) is “pulling” x towards 0 but g(x) is needed to obtain sparse solutions.

In embodiments, the step 230 may be executed once with the penalty factor g(x) to generate a first optimum candidate vector x_{opt_1} with the lowest error and then execute step 230 again without the penalty factor g(x) but by imposing:

$$∀ j ∈ \{ 1 , … , a \} {x}_{opt \_ 1 , i} = 0 ⇒ {x}_{i} = 0$$

meaning that, in the second execution of step 230, the search is performed with candidate vectors having a reduced length, where only the non-zero components of the first optimum candidate vector are considered.

The dimension of the search multidimensional space is thus reduced: the initial mesh including second candidate vectors having the reduced length is generated and the search is performed in a multidimensional space of reduced dimension to find a second optimum candidate vector xopt_2 with the lowest error that gives the quantitative contribution values of the associated antibody targets.

In these embodiments, the first execution of step 230 allows to identify the positive antibody targets: they correspond to the non-zero components of the first optimum candidate vector x_{opt_1}. While the second execution of step 230 allows computing the quantitative contribution values of the antibody targets identified with the first execution of step 230.

Because the set of solutions is discrete, generating all possible candidate vectors in the multidimensional space should ideally be evaluated. Unfortunately, doing this would lead to typically 200×25000³⁵⁵ operations which is around 3.7130×10¹⁵⁶³ operations to analyse only one serum.

To reduce the number of operations and the processing time in complex cases with many possible antibody targets, the set of candidate vectors to be evaluated may be reduced in several manners.

A first reduction can be achieved by only considering a reduced set of eplets for defining the set of antibody targets so that at least each antigen has an eplet in this minimum set of eplets. Then, applying the algorithm to compute the quantitative contributions of these eplets and checking if any antigens real MFI is not too far from the sum of the contributions (with a threshold that may be adjusted by the user). If the difference is too high for at least one antigen, eplets may be added to the set of antibody targets and the algorithm is run again with this increased set of eplets until no real MFI is too far from the sum of the contributions determined by the algorithm or until there is no more eplets to add to the set of antibody targets.

A second reduction can be achieved by enforcing a value on a given eplet, effectively reducing the problem's dimension.

Each of FIGS. 4A-4F shows a table with simplified experimental results obtained for a simplified case according to an example. Each table includes 3 columns. The first column includes the list of antigens that have been tested in the serum of a patient. The second column includes the antibody targets associated with each tested antigen and used as input to the algorithm. The third column includes the measurement values (here MFIs) obtained for the antigens.

In the example of FIG. 4A, the algorithm identifies as output the antibody against the antibody target DQ3 with an associated contribution to the MFI equal to 1000.

In the example of FIG. 4B, the set of antigens is the same as in FIG. 4A but the measurements are performed with the uncertainty of the MFIs inherent to any experimental procedure. Here also the algorithm identifies as output the antibody against the antibody target DQ3 with an associated contribution to the MFI equal to 1010.

In the example of FIG. 4C, the algorithm identifies as output two antibodies: an antibody against the antibody target DQ3 with an associated contribution to the MFI equal to 1000 and a specific antibody against the antibody target DQ8 with an associated contribution to the MFI equal to 4000.

In the example of FIG. 4D, that includes a different measurement value for the second and sixth antigens (1200 instead of 5000) compared to FIG. 4C, the algorithm identifies as output only one antibody against the antibody target DQ3 with an associated contribution to the MFI equal to 1000. The difference between the DQ3 that are not of the DQ8 antigenic subtype (i.e. DQ7 and DQ9) and the DQ8 is not statistically significant (especially when adding the uncertainty of the experimentally generated MFIs).

In the example of FIG. 4E, the algorithm identifies as output only one antibody against the antibody target DQ3 with an associated contribution to the MFI equal to 16000. The difference between the DQ3 that are not of the DQ8 subtype (i.e. DQ7 and DQ9) and the DQ8 is not statistically significant (especially when adding the uncertainty of the experimentally generated MFIs).

In the example of FIG. 4F, the algorithm identifies as output two antibodies: an antibody against the alpha chain QA3 antigen with an associated contribution to the MFI equal to 6000 and an antibody against the beta chain DQ3 antigen with an associated contribution to the MFI equal to 1000.

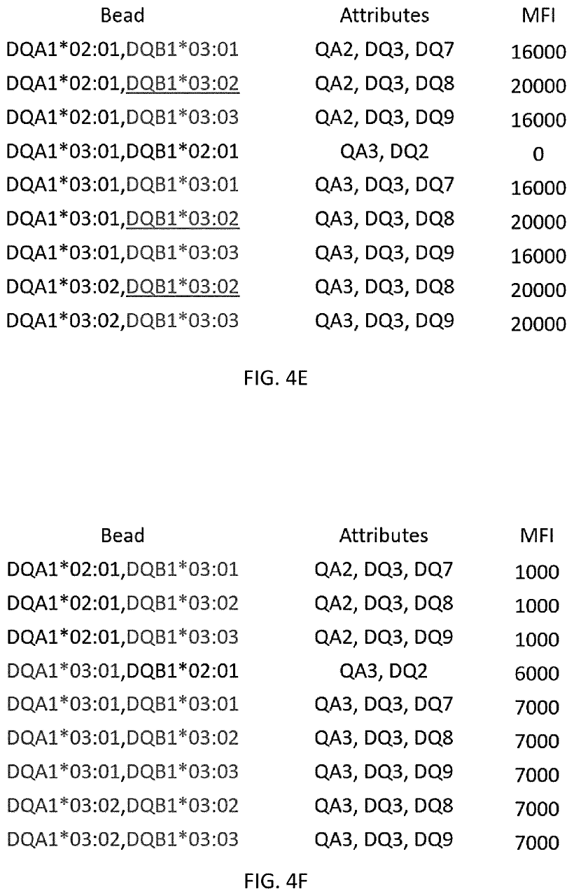

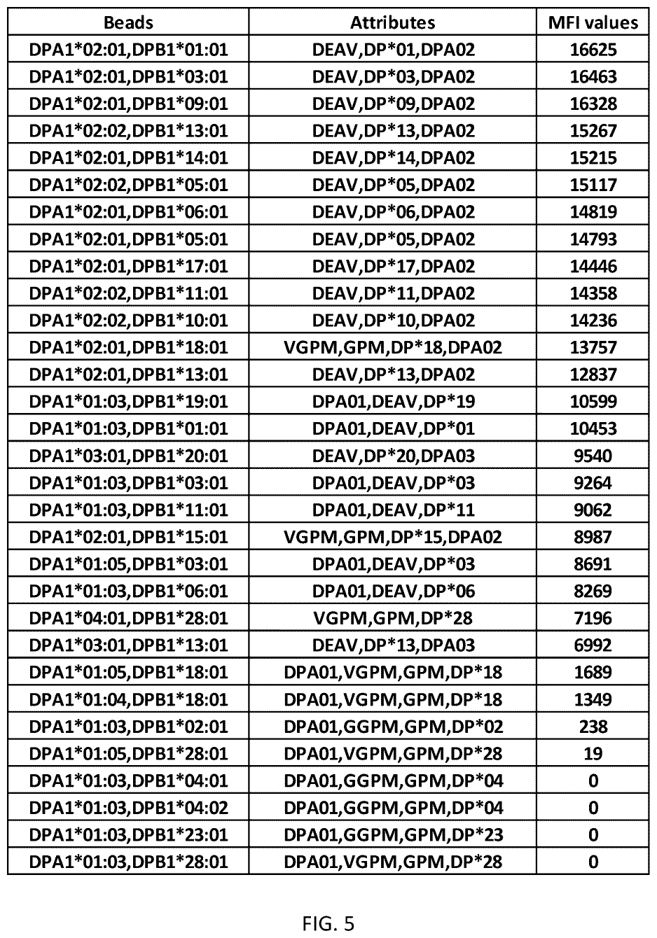

FIG. 5 shows a table with real experimental results obtained for a slightly more complex case. The table of FIG. 5 shows an anti-HLA-DP antibody profile from a patient serum.

With this antibody profile, 25 beads out of a total of 31 are positive and the antibody profile is explained by 3 antibodies (DEAV, DPA02 and DP*18): the algorithm provides as output the quantitative contributions to the MFI (thus the “strength”) equal to 7912, 7716 and 2512 respectively. The algorithm establishes the best compromise to explain the maximum number of positive beads while respecting each measured MFI.

The computation time was 23 seconds and there was only one unexplained bead (the bead carrying the antigen: DPA1*04:01DPB1*28:01). The positivity of this bead is indeed unexplainable with the proposed input since none among the VGPM, GPM and DP*28 antibody targets can be involved in the explanation of the complete antibody profile, each of them being present on at least one bead with negative MFI. This could mean that the list of antibody targets provided to the algorithm is incomplete, either because not updated recently enough, or because the current knowledge of antibody targets for DP antigens is not exhaustive at the time of the experiment.

The antibody targets used to solve this antibody profile are deliberately very few, but already the manual interpretation currently carried out for clinical use does not go that far as it does not consider the well-recognized eplets DEAV, VGPM, GPM, etc. . . . Although being eplets, these antibody targets are used mixed to antigens (DPxx for the DP beta antigens and PAxx for the DP alpha antigens), as they present the interest of gathering a large number of antigens sharing a common eplet easily recognized in patients' sera profiles when present. If a more detailed interpretation is required, up to the full eplet stage, after discarding the antigenic antibody targets, the algorithm may use all or part of the 62 DP eplets referenced in https://www.epregistry.com.br depending on what the biologist selects.

The algorithm disclosed herein allows calculating the amount of measurement value (e.g., MFI) caused by each antibody in the serum. The algorithm allows obtaining an unparalleled accuracy of interpretation.

The algorithm may be implemented by a software application configured to obtain (e.g., from a database including mapping between antigens and antibody targets on the respective antigen) the subsets of antibody targets corresponding respectively to the antigens for which measurement values are received. The software application may include a user interface to allow a user viewing and/or updating and/or storing these subsets of antibody targets mapped to the antigens.

The software application may include a user interface to display computation results to a user. The computation results may include the identified antibody targets and their respective quantitative contribution to the measurement values. The computation results may include other computation results such as for example: an identification of antibody targets for which there was no reactive antibody, an antibody target that was not searched by the algorithm because the positivity of the bead(s) with this antibody target is explained by a broader attribute.

The software application may be configured to receive as input the patient's own antigens (HLA typing) and identify the positive antibodies/antibody target of the serum by removing the antibody targets belonging to the patient who provided the serum (as individuals do not make antibodies against themselves).

The software application may be configured to receive as input a donor's own antigens (HLA typing) and to identify the antibody targets that explain the profile that would belong to the transplant donor.

The software application may be configured to read data from a SQL database and write results in the same database.

The HLA typing of the recipient and/or donor being optional features, it is possible to obtain a “raw” interpretation of the antibody profile without this information.

It should be appreciated by those skilled in the art that any functions, engines, block diagrams, flow diagrams, state transition diagrams, flowchart and/or data structures described herein represent conceptual views of illustrative circuitry embodying the principles of the invention. Similarly, it will be appreciated that any flow charts, flow diagrams, state transition diagrams, pseudo code, and the like represent various processes.

Although a flow chart may describe operations as a sequential process, many of the operations may be performed in parallel, concurrently or simultaneously. Also some operations may be omitted, combined or performed in different order. A process may be terminated when its operations are completed but may also have additional steps not disclosed in the figure or description. A process may correspond to a method, function, procedure, subroutine, subprogram, etc. When a process corresponds to a function, its termination may correspond to a return of the function to the calling function or the main function.

Each described function, engine, block, step described herein can be implemented in hardware, software, firmware, middleware, microcode, or any suitable combination thereof.

When implemented in software, firmware, middleware or microcode, instructions to perform the necessary tasks may be stored in a computer readable medium that may be or not included in a computing device. The instructions may be transmitted over the computer-readable medium and be loaded onto the computing device. The instructions are configured to cause the computing device to perform one or more functions disclosed herein. For example, as mentioned above, according to one or more examples, at least one memory may include or store instructions, the at least one memory and the instructions may be configured to, with at least one processor, cause the computing device to perform the one or more functions. Additionally, the processor, memory and instructions, serve as means for providing or causing execution by the device of one or more functions disclosed herein.

The computing device may be a general-purpose computing device, a special purpose computing device, a programmable processing device, a machine, etc. The computing device may be or include or be part of: a user equipment, client device, mobile phone, laptop, computer, data server, computer, cloud-based server, web server, application server, proxy server, etc.

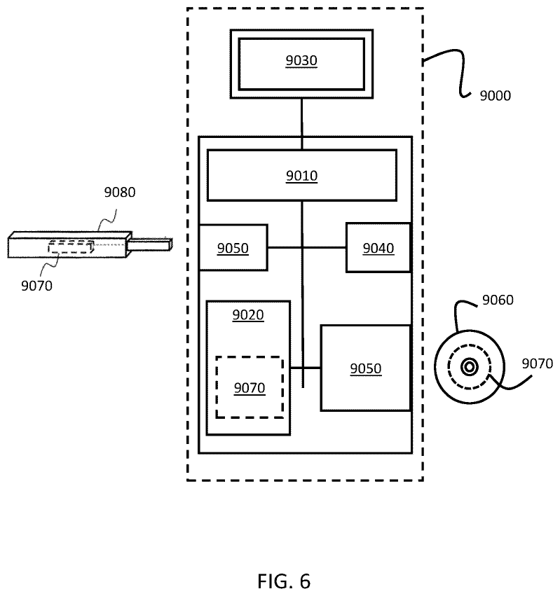

FIG. 6 illustrates an example embodiment of a computing device 9000. The computing device 9000 may be used for performing one or more or all steps of any method disclosed herein.

As represented schematically, the computing device 9000 may include at least one processor 9010 and at least one memory 9020. The computing device 9000 may include one or more communication interfaces 9040 (e.g., network interfaces for access to a wired/wireless network, including Ethernet interface, WIFI interface, etc) connected to the processor and configured to communicate via wired/non wired communication link(s). The computing device 9000 may include user interfaces 9030 (e.g., keyboard, mouse, display screen, etc) connected with the processor. The computing device 9000 may further include one or more media drives 9050 for reading a computer-readable storage medium (e.g., digital storage disc 9060 (CD-ROM, DVD, Blue Ray, etc), USB key 9080, etc). The processor 9010 is connected to each of the other components 9020, 9030, 9040, 9050 in order to control operation thereof.

The memory 9020 may include a random-access memory (RAM), cache memory, non-volatile memory, backup memory (e.g., programmable or flash memories), read-only memory (ROM), a hard disk drive (HDD), a solid-state drive (SSD) or any combination thereof. The ROM of the memory 9020 may be configured to store, amongst other things, an operating system of the computing device 9000 and/or one or more computer program code of one or more software applications. The RAM of the memory 9020 may be used by the processor 9010 for the temporary storage of data.

The processor 9010 may be configured to store, read, load, execute and/or otherwise process instructions 9070 stored in a computer-readable storage medium 9060, 9080 and/or in the memory 9020 such that, when the instructions are executed by the processor, causes the computing device 9000 to perform one or more or all steps of a method described herein for the concerned computing device 9000.

The instructions may correspond to program instructions or computer program code. The instructions may include one or more code segments. A code segment may represent a procedure, function, subprogram, program, routine, subroutine, module, software package, class, or any combination of instructions, data structures or program statements. A code segment may be coupled to another code segment or a hardware circuit by passing and/or receiving information, data, arguments, parameters or memory contents. Information, arguments, parameters, data, etc. may be passed, forwarded, or transmitted via any suitable technique including memory sharing, message passing, token passing, network transmission, etc.

When provided by a processor, the functions may be provided by a single dedicated processor, by a single shared processor, or by a plurality of individual processors, some of which may be shared. The term “processor” should not be construed to refer exclusively to hardware capable of executing software and may implicitly include one or more processing circuits, whether programmable or not. A processor or likewise a processing circuit may correspond to a digital signal processor (DSP), a network processor, an application specific integrated circuit (ASIC), a field programmable gate array (FPGA), a System-on-Chips (SoC), a Central Processing Unit (CPU), an arithmetic logic unit (ALU), a programmable logic unit (PLU), a processing core, a programmable logic, a microprocessor, a controller, a microcontroller, a microcomputer, a quantum processor, any device capable of responding to and/or executing instructions in a defined manner and/or according to a defined logic. Other hardware, conventional or custom, may also be included. A processor or processing circuit may be configured to execute instructions adapted for causing the computing device to perform one or more functions disclosed herein for the computing device.

A computer readable medium or computer readable storage medium may be any tangible storage medium suitable for storing instructions readable by a computer or a processor. A computer readable medium may be more generally any storage medium capable of storing and/or containing and/or carrying instructions and/or data. The computer readable medium may be a non-transitory computer readable medium. The term “non-transitory”, as used herein, is a limitation of the medium itself (i.e., tangible, not a signal) as opposed to a limitation on data storage persistency (e.g., RAM vs. ROM).

A computer-readable medium may be a portable or fixed storage medium. A computer readable medium may include one or more storage device like a permanent mass storage device, magnetic storage medium, optical storage medium, digital storage disc (CD-ROM, DVD, Blue Ray, etc), USB key or dongle or peripheral, a memory suitable for storing instructions readable by a computer or a processor.

A memory suitable for storing instructions readable by a computer or a processor may be for example: read only memory (ROM), a permanent mass storage device such as a disk drive, a hard disk drive (HDD), a solid state drive (SSD), a memory card, a core memory, a flash memory, or any combination thereof.

In the present description, the wording “means configured to perform one or more functions” or “means for performing one or more functions” may correspond to one or more functional blocks comprising circuitry that is adapted for performing or configured to perform the concerned function(s). The block may perform itself this function or may cooperate and/or communicate with other one or more blocks to perform this function. The “means” may correspond to or be implemented as “one or more modules”, “one or more devices”, “one or more units”, etc. The means may include at least one processor and at least one memory including computer program code, wherein the at least one memory and the computer program code are configured to, with the at least one processor, cause a computing device to perform the concerned function(s).

The term circuitry may cover digital signal processor (DSP) hardware, network processor, application specific integrated circuit (ASIC), field programmable gate array (FPGA), etc. The circuitry may be or include, for example, hardware, programmable logic, a programmable processor that executes software or firmware, and/or any combination thereof (e.g., a processor, control unit/entity, controller) to execute instructions or software and control transmission and receptions of signals, and a memory to store data and/or instructions.

Although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, when the term “and/or” is used in a list of items, it implies that the list may include any and all combinations of one or more of the associated listed items.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

While aspects of the present disclosure have been particularly shown and described with reference to the embodiments above, it will be understood by those skilled in the art that various additional embodiments may be contemplated by the modification of the disclosed machines, systems and methods without departing from the scope of what is disclosed. Such embodiments should be understood to fall within the scope of the present disclosure as determined based upon the claims and any equivalents thereof.

1. A method for identifying, in a set of antibody targets, one or more antibody targets corresponding to one or more antibodies in a serum, the method comprising:
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen; and
(d) identifying one or more antibody targets corresponding to one or more non-zero components in a candidate vector associated with the lowest error and extracting the non-zero components corresponding to the quantitative contributions of the identified one or more antibody targets.

2. The method of claim 1, wherein searching in the multidimensional search space the candidate vector associated with the lowest error comprises:
(c1) initializing candidate vectors forming an initial mesh;
(c2) repeating iteratively the following steps until at least one convergence criterion is met:
(c21) for each candidate vector, computing the associated error;
(c22) selecting candidate vectors with the lowest errors;
(c23) generating new candidate vectors based on the selected candidate vectors, the new candidate vectors forming a refined mesh in the multidimensional search space; and
(c3) selecting the candidate vector with the lowest error.

3. The method of claim 2, further comprising: eliminating from the set of measurement values each measurement value being below a detection threshold and eliminating from the set of antibody targets the one or more antibody targets present on at least one of the antigens for which the measurement value is below the detection threshold before performing step (c1).

4. The method of claim 2, further comprising eliminating each candidate vector including at least one component whose value is below a positivity threshold but non-zero, before performing step (c21) or (c22).

5. The method of claim 1, comprising:
identifying, in the set of antigens, independent groups of antigens such that there is no antibody target present on an antigen in a group that is also present on another antigen in another group; and
performing the steps (c) and (d) independently for each of the independent groups of antigens.

6. The method of claim 1, further comprising: adding a penalty factor to the sum of squared antigen errors, wherein the penalty factor increases as a function of the number and/or the value of the one or more non-zero quantitative contributions of the antibody targets in a candidate vector.

7. The method of claim 6, wherein the penalty factor is computed using a subadditive function of the candidate vector.

8. The method of claim 6, wherein the penalty factor is relaxed by:
executing steps (c) and (d) with the penalty factor to identify the non-zero components of the candidate vector with the lowest error; and
repeating steps (c) and (d) without the penalty factor with the candidate vectors being reduced to the identified non-zero components to determine a second candidate vector with the lowest error that gives the values of the identified non-zero components.

9. The method of claim 2, wherein the candidate vectors forming the initial mesh in the multidimensional space have a uniform spatial distribution.

10. The method of claim 2, wherein the candidate vectors forming the refined mesh in the multidimensional space have a uniform spatial distribution.

11. The method of claim 2, wherein a first convergence criterion is met when the elapsed time for executing step (c2) is greater than a threshold.

12. The method of claim 2, wherein a second convergence criterion is met when the cell dimensions of the smallest cells of the refined mesh reach a predefined limit.

13. (canceled)

14. A computing device comprising
at least one processor;
at least one memory storing instructions that, when executed by the at least one processor, cause the computing device to identify, in a set of antibody targets, one or more antibody targets corresponding to one or more antibodies in a serum, by
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen; and
(d) identifying one or more antibody targets corresponding to one or more non-zero components in a candidate vector associated with the lowest error and extracting the non-zero components corresponding to the quantitative contributions of the identified one or more antibody targets.

15. A non-transitory computer-readable medium comprising program instructions stored thereon for causing a computing device to identify, in a set of antibody targets, one or more antibody targets corresponding to one or more antibodies in a serum, by:
(a) obtaining input data including a set of measurement values obtained by solid phase assays for antibody identification for a set of antigens adsorbed on the solid phase and incubated in the serum, wherein a measurement value obtained for an antigen is representative of an amount of one or more antibodies bound to the solid phase and directed against one or more antibody targets present on the antigen;
(b) obtaining, for each antigen in the set of antigens, a subset of one or more antibody targets present on the antigen;
(c) searching in a multidimensional search space a candidate vector associated with the lowest error, wherein a candidate vector includes a component for each antibody target in the set of antibody targets and a component represents a quantitative contribution to a measurement value of the corresponding antibody target, wherein the error associated with a candidate vector is based on a sum of squared antigen errors across the set of antigens, wherein an antigen error is obtained by comparing the sum of quantitative contributions in the candidate vector with the measurement value obtained for the concerned antigen; and
(d) identifying one or more antibody targets corresponding to one or more non-zero components in the candidate vector associated with the lowest error and extracting the non-zero components corresponding to the quantitative contributions of the identified one or more antibody targets.
